(19) United States
(12) Patent Application Publication
(10) Pub. No.: US 2009/0042950 A1
(43) Pub. Date: Feb. 12, 2009

(54) TRANSDERMAL TOPICAL COMPOSITION AND ITS USES

(75) Inventor: Anant K. Pandya, Surrey (GB)

Correspondence Address: CATALYST LAW GROUP, APC, 9710 SCRANTON ROAD, SUITE S-170, SAN DIEGO, CA, 92121, US

(73) Assignee: Transphase Limited, Jersey, Channell Islands (GB)

(21) Appl. No.: 11/909,061

(22) PCT Filed: Mar. 22, 2006
(86) PCT No.: PCT/GB2006/001063

§ 371 Date: May 2, 2008

(30) Foreign Application Priority Data
Mar. 24, 2005 (GB) 0506139.5

(51) Int. Cl. A61K 47/24; A61K 47/32; A61K 31/465; A61K 47/10

(52) U.S. Cl. 514/343; 514/772.3; 514/770; 514/772

(57) ABSTRACT

At least one silicone is used to modify the release profile of the active compound(s) in a therapeutic composition for topical application the skin that also comprises at least one penetration enhancer and a fugitive solvent base.

The present invention relates to therapeutic compositions for topical application to the skin for transdermal delivery comprising at least one active compound. In particular, the invention relates to the use of at least one silicone in the compositions to modify transdermal delivery of the active compound(s).

The skin is the largest organ of the human body. It has an important role in protecting the body from mechanical injury, water loss and the entry of harmful agents (e.g. disease-causing bacteria). It is also a sensory organ, containing receptors-sensitive to pain, temperature and pressure. In warm-blooded animals, it helps regulate body temperature.

The skin is composed of two layers, the epidermis and the dermis. The epidermis has three layers, the outermost of which is called the stratum corneum which is a layer of dead keratinised cells forming a water-resistant barrier between the external environment and the living cells of the skin. The stratum corneum provides the first and most significant barrier to ingress of agents, for example pharmaceutically active agents, through the skin. In addition, the skin is constantly regenerating which makes prolonged application of such agents difficult.

The concept of administering drugs transdermally for a systemic effect is well established. The transdermal route of administration offers several advantages over the oral route using conventional preparations such as tablets or liquids. For example, transdermal delivery of a drug into the blood stream usually provides a constant level of the drug over a prolonged period of time. In addition, there is generally a low fluctuation in the level of the drug in the blood. Further, transdermal delivery not only avoids passage of the drug through the hostile environment of the gastro-intestinal tract and but also avoids hepatic first pass metabolism thereby increasing the bioavailability of the drug and reducing the dosage of the drug that has to be administered. Lower doses of the drug are possible which is especially advantageous for drugs which are poorly absorbed or undergo extensive first pass metabolism. Ease of use of transdermal preparations generally results in good patient compliance.

Existing transdermal formulations targeted for systemic delivery of active compounds can be classified broadly into four classes:
(i) semi-solid preparations such as creams, ointments, pastes, lotions or viscous dispersions;
(ii) liquid/gel reservoirs of drug sealed in a laminate;
(iii) semi-solid matrices in which the drug is dispersed and which have a peripheral adhesive; and
(iv) drug in adhesive.
   There are variations and combinations within these four classes.

A transdermal “patch” typically consists of a matrix or reservoir containing the drug to be administered, together with a backing layer, an adhesive and a protective release liner. Release membranes may also be incorporated. The delivery of drugs through these systems is either through passive diffusion, controlled by a semi-permeable release membrane, or is controlled by the adhesive/adhesive matrix. The system may also incorporate drug penetration enhancers to increase the flux of the drug through the skin.

One of the drawbacks of the current approaches is that the formulations are typically in continuous contact with the skin. Creams and ointments or adhesives used in patches can cause skin irritation and sensitisation. In this connection, it is estimated that 40% of patch users suffer from skin irritation and sensitisation due to adhesives used in the patch. There is a need, therefore, for an alternative topical method for the systemic administration of active compounds in which the irritancy potential is reduced.

A further disadvantage of creams and ointments is that they can leave an oily or greasy residue on the surface of the skin which frequently comes into contact with clothing. Such contact reduces the effective dose applied and causes stains and/or greasiness on the skin and/or the clothes of the subject and on any other material with which the composition may come in contact. These factors affect patient morale and can result in patient non-compliance with use of a medication. Oily residues on the skin can also in some cases hinder drug absorption.

This problem has been partially addressed in the context of local administration of active compounds using the compositions disclosed in U.S. Pat. No. 4,820,724. This reference discloses a solvent carrier system for the topical application of pharmaceutically active compounds, e.g. antifungal agents. The solvent carrier system comprises a first solvent phase of a relatively high boiling solvent and a second solvent phase of a relatively low boiling solvent. When applied topically, the relatively low boiling solvent evaporates leaving a concentrated solution of the active in the relatively high boiling solvent. The increase in concentration of the active compound assists penetration of the active compound into the skin. U.S. Pat. No. 4,850,724 exemplifies the use of a composition comprising 1 wt % griseofulvin, 10 wt % benzyl alcohol, 40 wt % acetone and 50 wt % (sic) isopropyl alcohol in the local treatment of tinea pedis infection.

The efficacy of a treatment of a condition usually depends at least in part on the release profile of the drug into the blood stream. Optimisation of the release profile for the condition in question leads to a more efficacious treatment. For example, some conditions are better treated with a fast peak concentration of the drug in the blood to enable a quick effect. Other conditions require a substantially constant concentration of the drug in the blood to enable a sustained effect over a period of time. There is a need, therefore, for a simple system for use in the topical administration of drugs to enable systemic treatment of a condition that enables modification of the release profile of the drug to suit the condition.

Silicones (or “siloxanes”) are usually inert compounds having good biocompatibility. Silicones are generally grouped into two groups, those having cyclic structures and those having straight or branched chain structures and silicones from the one group are known to have different properties from those of the other group.

Cyclic silicones (or cyclomethicones), for example, evaporate quickly at body temperature. Therefore, they are suitable for use as carrier silicones as well as light degreasers for lotions and other emulsified products. Straight chain silicones (e.g. dimethicones) are known to have a wide variety of properties based upon the length of the chain. Relative chain length is often differentiated by viscosity.

Polydimethylsiloxanes are known for use as a base in a pharmaceutical composition for external use. For example, JP-A-59053408 (Satou et al; published 1984) discloses pharmaceutical compositions for external use comprising a drug compound (e.g. benzodiazepine or clondine) having a systemic therapeutic effect; a urea derivative (e.g. 1,3-dimethylurea) as a penetration enhancer; and a linear polydimethylsiloxane (e.g. hexamethyldisiloxane) or a cyclic polydimethylsiloxane (e.g. cyclooctamethyltetrasiloxane) having a specific viscosity as the base of the composition.

Certain siloxane derivatives are known to enhance transdermal drug penetration. For example, Akimoto et al (J. Controlled Release; 77; 2001; pp 49-57) discloses in vitro experiments demonstrating the use of oligodimethylsiloxanes containing a β-D-glucopyranosyl group and one chain end (Glc-ODMS) to enhance penetration of indomethacin or antipyrine through rat abdominal skin. The active compounds were applied to the rat skin in the form of an ethanolic aqueous solution (50 wt %).

EP-A-0484857 (Nagase et al; published 1992) discloses a group of polyorganosiloxanes having a quaternary salt at one end for use as low toxicity, low irritation penetration enhancers. The polyorganosiloxane salts were subjected to in vitro testing by administration of 2 ml of an aqueous 50% ethyl alcohol solution containing 20 mg (1 wt %) of antiphlogistic indometacin and 2 wt % of the polyorganosiloxane salt under examination to skin from the abdomen of a rabbit.

EP-A-0521607 (Colas et al; published 1993) discloses a group of carboxylalkyl functional polysiloxanes and alkylsulphoxide polysiloxanes for use as penetration enhancers causing lower irritation than conventional penetration enhancers. Ethanol solutions of a polysiloxane under examination at different concentrations were administered to the inner surface of mice ears to determine their irritancy.

The Inventors have discovered not only that the use of silicones facilitates systemic administration of active compound(s) through the skin using fugitive solvents but also that careful selection of the type, quantity, combination and proportion of the silicone(s) can optimise the release profile of the active compound(s) to suit the condition to be treated.

According to a first aspect of the present invention, there is provided a therapeutic composition for topical application to skin comprising:

at least one active compound;

at least one penetration enhancer;

a penetration modulating component comprising at least one silicone; and

a fugitive solvent base.

Compositions according to the present invention enable penetration of active compound(s) through the outermost keratin layer of the epidermis. In addition, these compositions deliver the desired amount of the active compound(s) in a controlled manner. Further, the compositions enable entry of the active compound(s) into the systemic circulatory system without leaving any residue on the skin or causing irritation, e.g. due to adhesive.

The release profiles for a therapeutic composition may be described in terms of the “Cₘₐₓ”, “Tₘₐₓ”, “Cₛₛ” and “Tₛₛ” values. The “Cₘₐₓ” value is the peak concentration of active compound(s). The “Tₘₐₓ” value is the period of time immediately after administration at which the peak concentration (or “Cₘₐₓ”) occurs. The “Cₛₛ”, value is the steady state (or constant) concentration of the active compound(s) where the rate of administration is equal to the rate of elimination. The “Tₛₛ” value is the period of time during which steady state concentration (or “Cₛₛ”) is maintained.

The Inventors discovered that there is an unexpected synergism between the penetration enhancer(s) and the silicone(s) of the penetration modulating component. For example, in some composition embodiments, the penetration modulating component increases Tₘₐₓ significantly (e.g. by over 500%) when compared to a control composition. A prolonged “steady state” of active compound(s) (Cₛₛ) is achieved in the blood over a greater Tₛₛ thereby sustaining release of the active compound(s). In other embodiments, the penetration modulating component increases Cₘₐₓ significantly (e.g. by over 250%) when compared to a control composition. Thus, the active compound(s) can be delivered with a fast peak concentration. Some embodiments even display increases in Tₘₐₓ, Cₘₐₓ and Cₛₛ.

The Inventors also discovered that, in some embodiments, the penetration modulating component increases substantially (e.g. by over 50%) the total amount of active component that is delivered over a 24 hour period when compared to a control composition. Thus, bioavailability of active compound(s) using compositions of the present invention may be increased not only over oral routes of administration but also over existing transdermal routes.

The control composition consists of all of the components of the composition of the present invention under investigation except for the penetration modulating component which is replaced with further fugitive solvent base.

The Inventors believe that the results are entirely unexpected and unpredictable in view of the prior art and indicate that the release profile of one or more active compounds may be controlled depending on the composition of silicone(s) in the penetration modulating component.

The penetration modulating component preferably comprises one or more silicones from the group consisting of polydimethylsiloxanes (e.g. dimethicones; and cyclomethicones) and oligodimethylsiloxanes (e.g. hexa-methyldisiloxane (“HMDS”) and octamethyltrisiloxane (“OMTS”)). Simethicones (i.e. dimethicones activated with silicon dioxide) may also be used.

Dimethicones are graded according to their viscosities. Suitable dimethicones may have a viscosity from about 20 centistokes (“cSt”) to about 1250 cSt, preferably from about 20 cSt to about 1000 cSt. Preferred dimethicones have a viscosity of about 20 cSt, about 100 cSt or about 350 cSt. The most preferred dimethicone is either Dimethicone USP NF or Dimethicone Ph.Eur. The grading for cyclomethicones is less well defined. The preferred cyclomethicone is Cyclomethicone USP NF or Cyclomethicone Ph.Eur.

The penetration modulating component is typically present in the composition in an amount of from about 10 wt % to about 70 wt % based on the total weight of the composition.

The penetration modulating component preferably consists essentially of either a single silicone or a combination of silicones, particularly a mixture of two or three silicones. The use of different silicones and combination of silicones has been found to change the release profile of the active compound(s). This surprising and unexpected discovery gives rise to a number of different composition embodiments characterised by the silicone content and resultant release profile(s) of the active compound(s).

The penetration modulating component may comprise cyclomethicone. The penetration modulating component may consist essentially of cyclomethicone alone or may further consist of either dimethicone or an oligidimethylsiloxane having a high volatility such as HMDS or OMTS. Additionally, the penetration modulating component may consist essentially of cyclomethicone with dimethicone and at least one oligodimethylsiloxane such as HMDS or OMTD. Alternatively, the penetration modulating component may consist essentially of dimethicone and at least one oligomethyldisiloxane such as HMDS or OMTS without cyclomethicone. The release profiles resulting from the use of cyclomethicone alone and from these particular combinations of silicones are different. The differences and their significance are discussed below.

In a first embodiment, the penetration modulating component consists essentially of cyclomethicone. When used alone, the cyclomethicone is preferably present in an amount from 20 wt % to 40 wt % and preferably about 30 wt % based on the total weight of the composition.

In a second embodiment, the penetration modulating component consists essentially of cyclomethicone, dimethicone, and at least one oligodimethylsiloxane, particularly HMDS. In this embodiment, cyclomethicone is usually present in an amount of from about 20 wt % to about 40 wt %, preferably about 30 wt %, based on the total weight of the composition. Dimethicone is usually present in an amount of from about 5 wt % to about 30 wt %, preferably about 10 wt %, based on the total weight of the composition. The oligodimethylsiloxane (e.g. HMDS) is usually present in an amount of from about 15 wt % to about 25 wt %, preferably about 20 wt %, based on the total weight of the composition.

In a third embodiment, the penetration modulating component consists essentially of cyclomethicone and dimethicone. In this embodiment, cyclomethicone is usually present in an amount of from about 15 wt % to about 35 wt %, preferably 30 wt %, based on the total weight of the composition. Dimethicone is usually present in an amount of from about 5 wt % to about 30 wt %, preferably 10 wt %, based on the total weight of the composition.

In a fourth embodiment, the penetration modulating component consists essentially of cyclomethicone and at least one oligodimethylsiloxane, particularly HMDS. In this embodiment cyclomethicone is usually present in an amount of from about 15 wt % to about 25 wt %, preferably 20 wt %, based on the total weight of the composition. The oligodimethylsiloxane (e.g. HMDS) is usually present in an amount of from about 5 wt % to about 20 wt %, preferably 10 wt %, based on the total weight of the composition.

In a fifth embodiment, the penetration modulating component consists essentially of dimethicone and at least one oligodimethylsiloxane, particularly HMDS. In this embodiment, the dimethicone is usually present in an amount of from about 20 wt % to about 40 wt %, preferably about 30 wt %, based on the total weight of the composition. The oligodimethylsiloxane (e.g. HMDS) is usually present in an amount of from about 10 wt % to about 30 wt %, preferably about 20 wt %, based oh the total weight of the composition.

OMTS may be used in place of HMDS. In such embodiments, the proportion of OMTS would usually be the same as that for HMDS described above.

In some embodiments, a mixture of oligodimethyl-siloxanes such as HMDS and OMTS may be used together with either dimethicone or cyclomethicone. In other embodiments, such a mixture may be used together with both dimethicone and cyclomethicone. A preferred mixture of oligodimethyl-siloxanes is Dow-Corning® Q7-9180 Silicone Fluid 0.65 CST (Dow-Corning, Meriden Business Park, Copse Drive, Allesley, Coventry, CV5 9RG, UK) which is a mixture of HMDS and OMTS.

In the first, second and fifth embodiments, the penetration modulating component usually increases Cₘₐₓ for the therapeutic composition when compared to a control composition, at a Tₘₐₓ of typically less than 6 hours (for the first and second embodiments). The increase in Cₘₐₓ is usually significant, typically over 100% and often about 250% more than that for comparative compositions without the penetration modulating component.

The first and second composition embodiments in particular are suitable if a high peak concentration of active compound(s) is required quickly. These penetration modifying components also have the effect of increasing the total amount of active compound(s) that is delivered over a period not less than 24 hours relative to a corresponding composition without a penetration modulating component.

In the third, fourth and fifth embodiments, Tₘₐₓ for the therapeutic compositions is greater than Tₘₐₓ for a control composition. In addition, there is usually no substantial change in Cₘₐₓ for compositions using these penetration modulating components. The Tₘₐₓ value of the active compound(s) is usually increased by at least 100%, typically by at least 300% and often by at least 500%. In some embodiments, the Tₘₐₓ value is at least 16 hours and, in other embodiments, the Tₘₐₓ value may be over 24 hours. The Cₛₛ value remains constant (or at least substantially constant) over a period (or Tₛₛ) of at least 16 hours and, in other embodiments, the Cₛₛ value may remain constant (or at least substantially constant) for a period (or Tₛₛ) of over 24 hours.

The third, fourth and fifth embodiments are suitable for administering the active compound(s) in a sustained manner thereby achieving a substantially “steady state” of the active(s) (Cₛₛ) in the blood. Preferred compositions sustain release of the active compound(s) at or about Cₛₛ from about 6 hours to at least 24 hours.

The compositions of the present invention are suitable for use as vehicles for the topical application of specific compounds to the skin using pharmaceutical, nutraceutical, cosmetic or veterinary preparations. Such topical application enables the specific compounds to penetrate the skin and enter the circulatory system thereby enabling the active compound(s) to have a systemic effect.

The or at least one active compound may be a pharmacologically active compound. A “pharmacologically active compound” is a compound that has a therapeutic effect on the human or animal body in the treatment or prevention of a condition.

Suitable pharmacologically active compounds may be selected from:
H2-receptor antagonists such as cimetidine; and ranitidine;
Prostaglandin analogues such as misoprostol;
Proton pump inhibitors such as lansoprazole; omeprazole; and pantaprazole;
Agents to treat food allergies such as sodium cromoglicate;
Cardiac glycosides such as digoxin;
Diuretics such as amiloride; bendroflumethizide; indapamide; furosemide; hydrochlorothiazide; and xipamide;
Drugs for arrythmias such as procainamide; lidocaine; propranolol; atenolol; bisoprolol; carvedilol; pindolol; and nebivolol;
Antihypertensives and agents for treatment of angina such as clizapril; lisinopril; ramipril; trandolapril; amlodepine; losartan; glyceryl trinitrate; isosorbide mononitrate; diltiazem; felodipine; isradipine; and lacidipine;
Lipid regulating drugs such as statins;
Drugs acting on the respiratory system such as salbutamol; terbutaline; and bambuterol;
Antihistamines such as cinnarazine; promethazine; perphenazine; and prochlorprazine;
Hypnotics such as zolpidem; zopiclone; and clomethiazole;
Anxiolytics such as benzodizapines; and buspirone;
Antipsychotic agents such as benperidol; fluphenazine; pimozide; and amisulpride;
Antidepressant drugs such as tricyclics; mianserin; and MAOIs;
Seretonin re-uptake inhibitors such as reboxetine;
Central nervous system stimulants such as methylphenidate;
Drugs used in the treatment of nausea such as antihistamines; domperidone; metoclopramide; 5HT₃ antagonists; hyoscine; and betahistine;
Opioid analgesics such as morphine; buprenorphine; and fentanyl;
Anti-migraine drugs such as 5HT₁ agonist and ergot alkaloids;
Drugs used in treatment of Parkinsonism such as apomorphine; bromocriptine; lisuride; haloperidol; and ergot alkaloids;
Drugs used in substance dependence such as nicotine and buprenorphine;
Drugs used in dementia such as rivastigmine; dihydroergotamine; dihydroergocristine; and dihydroergocryptine.
Antibiotics; antifungals; antivirals and antimalarials
Drugs used in treatment of diabetes
Drugs for glucocorticoid therapy using steroids such as betamathasone and dexmethasone;
Male and female sex hormones such as estradiol; norethisterone; progesterone; testosterone; and their esters;
Pituitary hormones such as vasopressin and desmopressin;
Drugs affecting bone metabolism such as calcitonin and bisphosphonates;
Endocrine drugs such as bromocriptine and cabergoline;
Contraceptives such as oestrogens; progestrogens and combinations thereof;
Drugs used in urinary frequency and enuresis such as oxybutinin and desmopressin;
Drugs used in erectile dysfunction such as apomorphine and sildenafil;
Drugs used in malignant disease and immunosuppression such as buslfan; antimetabolites; alkaloids; corticosteroids; hormones and interferons;
Non-steroidal anti-inflammatory drugs such as diclofenac; piroxicam and refoxicab;
Drugs used in treatment of gout such as colchicines;
Drugs used in neuromuscular disorders such as neostigmine and pyridostigmine;
Muscle relaxants such as diazepam; and tizanidine;
Vaccines delivered by subcutaneous route; and
Agents for the treatment of nicotine withdrawal symptoms such as nicotine.

The or at least one active compound may be a nutraceutically active compound. A “nutraceutically active compound” is a compound, derived from a natural origin (animal or vegetable) that has a beneficial and/or therapeutic effect on the human or animal body in the treatment of a condition. Such compounds may be regarded as nutrients.

Suitable nutraceutically active compounds may be natural products extracted from animals or vegetables. Examples of suitable nutraceutically active compounds include:
carotenoids such as lycopene, lutein, astaxanthin and β-carotene;
glucosamine or N-acylglucosamine;
ubiquinone;
Vitamins such as vitamins A, C, D and E;
Rosmarinic acid;
Honokiol;
Magnolol;
Chlorogenic acid;
Oleuropein;
Methylsulphonylmethane (“MSM”);
Collagen and Chondroitin;
Boswellin and boswellic acid;
Escin and esculin;
Tumeric extracts such as curcuminoids and tetrahydrodurcuminoids;
Gingerol and gingerone;
Triterpenes such as ursolic acid and oleanolic acid;
Diterpenes such as asiaticoside, sericoside and ruscogenins;
Hydroxycitric acid (“HCA”) and niacinamide hydroxycitrate; Trigonellin;
Corosolic acid;
Saw palmetto; and
St John's Wort.

Pharmacologically acceptable derivatives (including salts) of the pharmacologically or nutraceutically active compounds may also be used.

The composition may comprise one or more components having a cosmetic effect. Such components include collagen and retinols.

The pharmacologically active compounds, the nutraceutically active compounds and the cosmetic components may either be used alone or in any combination.

The active compound is present in preferred embodiments in a therapeutic amount, e.g. an amount calculated to enable a beneficial and/or therapeutic effect on the human or animal body with the correct dosage. The active compound(s) is typically present in an amount of from about 0.1 wt % to about 10 wt % based on the total weight of the composition. In some preferred embodiments, the amount is from about 0.5 wt % to 5 wt % and more preferably from about 1 wt % to about 3 wt %, for example about 1 wt % or about 2 wt %.

Without wishing to be bound by any particular theory, the inventors believe that embodiments of the invention resulting in sustained release of the active compound(s) work by depositing a “depot” of the active compound(s) under the skin. In the third, fourth and fifth embodiments at least, the or each active compound is then released at a substantially constant rate from the depot to achieve a substantially steady state of active compound(s) in the blood.

Preferred compositions are non-aqueous.

Some silicones, e.g. dimethicone, have emollient properties and, thus, the compositions of the present invention have less irritancy potential than corresponding compositions without a silicone-based penetration modulating component. This advantage is particularly apparent in compositions comprising an alcoholic fugitive solvent base which tend to have a higher irritancy potential, particularly when used on sensitive skin or skin that is split or raw or has lesions.

Where necessary, however, the compositions of the present invention may further comprise an emollient component. Such a component assists the silicone(s) in reducing the irritation potential of the compositions. The emollient component may be a single compound or a mixture of compounds. Suitable compounds for use in the emollient component include glycols (e.g. propylene glycol); polyglycols; fatty acids and their derivatives such as fatty acid esters; and vegetable oils.

The emollient component is typically present in an amount of from about 5 wt % to about 50 wt %, preferably from about 5 wt % to about 40 wt % and more preferably from about 5 wt % to about 35 wt %, calculated on the basis of the total weight of the composition. In preferred embodiments, the emollient component is present in an amount of about 5 wt % to about 20 wt % of the total composition.

Some silicones, particularly cyclomethicone, have penetration enhancing properties and thus may be used as the penetration enhancer of the present invention. However, where further penetration enhancement is required, the compositions may further comprise at least one non-silicone penetration enhancer. Examples of suitable non-silicone penetration enhancers for use in preferred compositions of the present invention include benzyl alcohol; azone; and triglyceride fatty acids. Benzyl alcohol is particularly preferred.

Where present, the penetration enhancer is typically present in an amount of from about 5 wt % to about 15 wt %, based on the total weight of the composition. In preferred embodiments, the penetration enhancer is present in an amount of about 10 wt %.

The purpose of the fugitive solvent base is to provide a medium by which the active(s) is administered to the skin and then to evaporate thereby driving the active(s) into skin and leaving a portion of the active(s) concentrated in the residue on the surface of the skin. The fugitive solvent base usually comprises an alcohol, preferably a C₁-C₄ alcohol. Monohydric aliphatic alcohols such as methyl alcohol; ethyl alcohol; propyl alcohol; isopropyl alcohol; and butyl alcohol are preferred. Isopropyl alcohol is particularly preferred. Mixtures of alcohols may also be suitable. For example, the fugitive solvent may consist of a mixture of isopropyl alcohol and ethyl alcohol.

Ketones, e.g. C₁-C₄ ketones such as acetone; propanone; and butanone, may also be present in the fugitive solvent base. Acetone is preferred. In some embodiments, the fugitive solvent base may consist of a mixture of monohydric aliphatic alcohol and a ketone. For example, the fugitive solvent base may consist of a mixture of isopropyl alcohol and acetone.

The choice of components for the fugitive solvent base depends on the stability of the active(s) in the composition. Salts of some active(s) react with ketones. For example, some nicotine metabolites react with acetone. Thus, ketones are not suitable components for the solvent base where the active is such a molecule. In such cases, a mixture of monohydric aliphatic alcohols might be used.

The mixture may comprise isopropyl alcohol and ethanol. The isopropyl alcohol may be present in an amount of from about 10 wt % to about 40 wt %, preferably about 25 wt 0% to about 35 wt % and most preferably about 30 wt %, based on the total weight of the composition. The ethanol may be present in an amount of from about 10 wt % to about 50 wt %, typically about 10 wt % to about 40 wt %, preferably about 25 wt % to about 35 wt % and most preferably about 30 wt %, based on the total weight of the composition.

In embodiments of the present invention in which the fugitive solvent base is a mixture of monohydric aliphatic alcohol (e.g. isopropyl alcohol) and ketone (e.g. acetone), the monohydric aliphatic alcohol is typically present in an amount of from about 20 wt % to about 50 wt % and preferably from about 25 wt 6 to about 40 wt %, based on the total weight of the composition. The ketone is typically present in an amount of from about 20 wt % to about 50 wt % and preferably from about 25 wt % to about 35 wt %, based on the total weight of the composition.

The compositions of the present invention may be in any form suitable for topical application to the skin. Suitable forms include sprayable liquids; gels; liquids that may be applied using a roll-on device; lacquers; and sustained release matrices of transdermal delivery devices such as patches. The compositions are usually administered alone but, under some circumstances, administration may be further modified by using other delivery mechanisms such as iontophoresism, ultrasound and microneedles to enhance penetration.

The compositions of the present invention have particular application in the topical administration of active compounds for a systemic effect.

Compositions of the present invention may be administered over a defined area using a suitable device. According to a second aspect of the present invention, there is provided a dispenser comprising a container containing a dispensable composition according to the first aspect and dispensing means for dispensing the composition. Preferably, the dispensing means dispenses a metered or measured dose of the composition. In such preferred embodiments, the dose may be metered or measured in terms of the weight of the formulation or in terms of the area covered. One advantage of these embodiments is that the risk of over or under dosing of the active(s) is reduced.

In one preferred embodiment, the composition is in the form of a sprayable liquid that may be administered using a spray dispenser. A suitable spray dispenser comprises a container containing a sprayable composition according to the first aspect and dispensing means suitable for dispensing the composition in the form of a spray.

In another preferred embodiment, the composition is in the form of a liquid that may be administered using a roll-on device. A suitable roll-on device comprises a container containing a liquid composition according to the first aspect and roller dispensing means suitable for dispensing the composition.

In other preferred embodiments, the composition is applied in the form of a lacquer.

According to a third aspect of the present invention, there is provided a therapeutic composition as defined in the first aspect for use in the treatment of the human or animal body by therapy.

According to a fourth aspect of the present invention, there is provided use of a penetration modulating component comprising at least one silicone in a therapeutic composition for topical application to the skin comprising:

at least one active compound;

at least one penetration enhancer; and

a fugitive solvent base,

to increase at least one value selected from Tₘₐₓ, Cₘₐₓ, Cₛₛ and Tₛₛ for said therapeutic composition relative to a control composition consisting of each of the components of said therapeutic composition except for the penetration modulating component.

Cyclomethicone may be used alone or in combination with dimethicone and HMDS (or OMTS). In such embodiments, Cₘₐₓ for the active compound(s) is usually increased, typically by at least 100%, at a Tₘₐₓ of less than 6 hours. The total amount of active compound that is delivered over a period not less than 24 hours may also be increased. Cₘₐₓ is also usually increased if dimethicone is used with HMDS in the absence of cyclomethicone.

Cyclomethicone may be used in combination with either dimethicone or at least one oligodimethylsiloxane (e.g. selected from HMDS and OMTS). Alternatively, dimethicone may be used in combination with at least one oligodimethylsiloxane (e.g. selected from HMDS and OMTS). In such embodiments, Tₘₐₓ of active compound(s) is increased without a substantial change in Cₘₐₓ. The increase in Tₘₐₓ of active compound(s) is usually at least 16 hours and may be over 24 hours. In these embodiments, release of the active compound(s) may be sustained to achieve a substantially steady state concentration (Cₛₛ) of active(s) in the blood from about 6 hours to at least 24 hours. The combination of cyclomethicone and HMDS (or OMTS) is particularly useful as it can provide substantially Cₛₛ for a period of over 24 hours without reaching Cₘₐₓ for the composition.

There is also provided a method of modifying transdermal penetration of the or each active compound in a therapeutic composition for topical application to skin comprising at least one active compound and a fugitive solvent base relative to a corresponding composition without a penetration modulating component, said method comprising using at least one silicone in said composition as a penetration modifying component.

There is also provided a method of preparing a therapeutic composition for application to skin comprising at least one active compound; at least one penetration enhancer; a penetration modulating component; and a fugitive solvent base, said method comprising:

determining an appropriate release profile for the or each active component in view of the condition to be treated;

selecting an appropriate silicone or combination of silicones as said penetration modifying component; and

combining the or each active compound with the fugitive solvent base, the or each penetration enhancer and said penetration modifying component.

Therapeutic compositions of the present invention may be used to treat or prevent a wide variety of conditions depending on the choice of active compound or combination of active compounds. Methods of treatment or prophylaxis of the conditions comprise administering topically to an area of skin a therapeutic amount of an appropriate composition according to the present invention. In this connection:
Gastric and peptic ulcers may be treated using H2-receptor antagonists such as cimetidine; and ranitidine; or using proton pump inhibitors such as lansoprazole; omeprazole; and pantaprazole;
Benign gastric and duodenal ulceration may be treated using prostaglandin analogues such as misoprostol;
Food allergies may be treated using agents such as sodium cromoglicate;
Heart conditions may be treated using cardiac glycosides such as digoxin;
Blood pressure and oedema may be controlled using diuretics such as amiloride; bendroflumethizide; indapamide; furosemide; hydrochlorothiazide; and xipamide;
Arrythmias may be treated using drugs such as procainamide; lidocaine; propranolol; atenolol; bisoprolol; carvedilol; pindolol; and nebivolol;
Hypertension and angina may be treated using antihypertensives such as clizapril; lisinopril; ramipril; trandolapril; amlodepine; losartan; glyceryl trinitrate; isosorbide mononitrate; diltiazem; felodipine; isradipine; and lacidipine; and
The symptoms of nicotine withdrawal may be treated with nicotine.
   Other conditions that may be treated using the present invention include those conditions that may be treated using any of the active compounds described above.

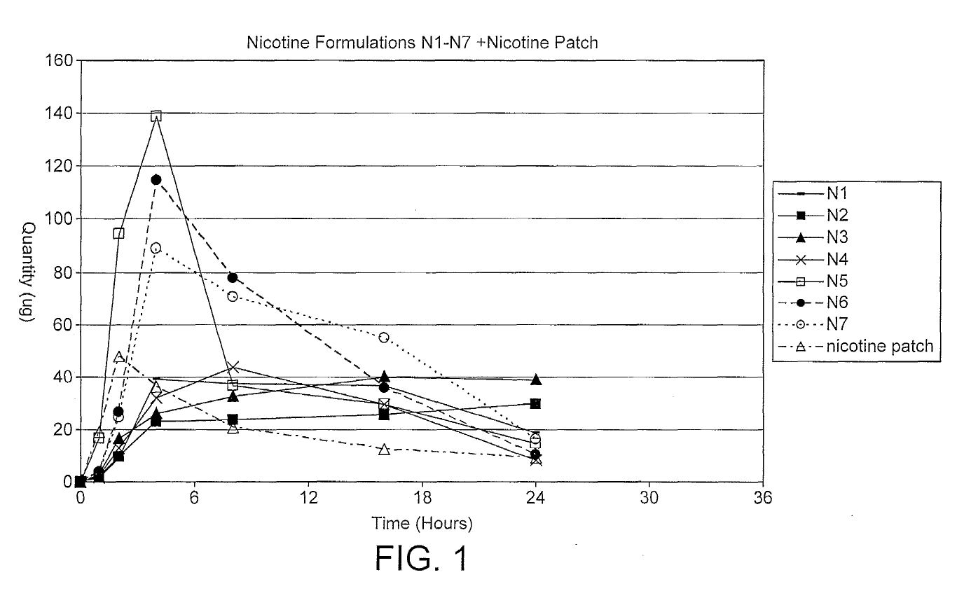

The following is a description, by way of example only and with reference to FIG. 1, of a presently preferred embodiment of the invention.

FIG. 1 is a graphical representation of the results of a study to compare in vitro dermal penetration of six nicotine-containing formulations according to the present invention (N2 to N7) with a control formulation (N1) and a commercially available nicotine matrix patch (NICORETTE (15 mg)).

### EXAMPLE

A study was performed to compare in vitro dermal penetration of seven nicotine-containing transdermal spray formulations (N1 to N7; for compositions, see Table 1) and a commercially available nicotine matrix “patch” (NICORETTE (15 mg); GlaxoSmithKline plc, 980 Great West Road, Brentford, Middlesex, TW8 9GS, UK) through dermatomed human skin (TRANSKIN) membranes.

Dermatomed human skin membranes were prepared and fitted inside flow through diffusion cells having a dose exposure area of 0.64 cm². The cells were placed in a temperature controlled heater block so that the cells were maintained at a constant temperature of 32° C.±2° C. 64 μl (100 μl/cm²) of the nicotine-containing formulations was topically applied to the membrane surface (up to six replicates). The nicotine matrix patch was subdivided to fit the flow through diffusion cells and applied to the membrane surface (up to six replicates). Receptor fluid (phosphate buffered saline) passed under the membranes was collected at timed intervals up to 24 hours post dosing. The fractions of receptor fluid collected at 0 hours (pre-dose) and 1, 2, 4, 8, 16 and 24 hours post dosing were analysed for nicotine content using HPLC in order to calculate the quantity (μg) of nicotine delivered across the human skin membranes for each formulation (N1 to N7) and the nicotine matrix patch.

The results of the study are indicated in Table 1 and depicted graphically in FIG. 1.

TABLE 1

| NICOTINE | | | | | | | | NICORETTE |
|---|---|---|---|---|---|---|---|---|
| FORMULATION | N1 | N2 | N3 | N4 | N5 | N6 | N7 | PATCH |
| Nicotine | 1 | 1 | 1 | 1 | 1 | 1 | 2 | — |
| Benzyl alcohol | 10 | 10 | 10 | 10 | 10 | 10 | 10 | — |
| Dimethicone USP | — | — | 10 | 30 | 10 | — | — | — |
| HMDS | — | 10 | — | 20 | 20 | — | — | — |
| Cyclomethicone | — | 20 | 30 | — | 30 | 30 | 30 | — |
| USP | | | | | | | | |
| Isopropyl alcohol | 45 | 30 | 24 | 20 | 15 | 30 | 29 | — |
| Ethanol | 44 | 29 | 25 | 19 | 14 | 29 | 29 | — |
| Amount Delivered | 418 | 220 | 346 | 348 | 584 | 638 | 607 | 253 |
| in 24 hour period | | | | | | | | |
| (μg) | | | | | | | | |
| Cₘₐₓ μg) | 25 | 20 | 26 | 28 | 89 | 74 | 57 | 30 |
| Tₘₐₓ (hours) | 4 | 24 | 16 | 8 | 4 | 4 | 4 | 2 |

(Data to nearesr whole number.)

The results clearly demonstrate that there are differences in the delivery profiles of nicotine from the transdermal spray formulations according to the present invention (N2 to N7), the control formulation (N1) and the nicotine matrix patch.

Over the 24 hour study, the overall delivery of nicotine through the skin membranes from formulations N3 to N7 (346, 348, 584, 638 and 607 μg respectively) was considerably greater than nicotine delivery from the commercially available nicotine matrix patch (253 μg). In addition, the overall delivery of nicotine through the skin membranes from formulations N5 to N7 (584, 638 and 607 μg respectively) was considerably greater than nicotine delivery from the control formulation N7 (418 μg).

The Cₘₐₓ values were greatest for formulations N5 to N7 (89, 74 and 57 μg respectively), for each of which the Tₘₐₓ value was 4 hours as compared with 2 hours for the nicotine matrix patch (Cₘₐₓ value 30 μg) and 4 hours for the control formulation N1 (Cₘₐₓ value 25 μg). These results indicate that the use of cyclomethicone alone (N6 and N7) and the use of the combination of cyclomethicone with both dimethicone and HMDS (N5) as the penetration modifying components results in a “high” and “fast” peak concentration of nicotine. Such release profiles are suitable for the rapid administration of a relatively large quantity of active compound.

The Tₛₛ values were the greatest for formulations N3 and N2 (16 hours and over 24 hours respectively). It is important to note that, for formulations N2 and N3, the rate of penetration of the nicotine during the period 6 to 24 hours is substantially constant at about Cₛₛ (20 and 26 μg respectively). Such release profiles are suitable for sustained administration of an active compound over a prolonged period of time at a substantially constant and relatively low rate of administration.

It should also be noted that Tₘₐₓ had not been reached for formulation N2 after 24 hours. Such a release profile may be suitable for sustained release of an active compound to achieve a substantially steady state (Cₛₛ) of the active compound in the blood for a prolonged period of time.

It may be concluded from the results that cyclomethicone promotes a “fast” (i.e. relatively small Tₘₐₓ), and “high” (i.e. relatively large Cₘₐₓ) dosing of active compound(s). When either dimethicone or HMDS is used in combination with cyclomethicone, administration of the active compound(s) is retarded. However, if both dimethicone and HMDS are used in combination with cyclomethicone, then a sustained dosing of active compound(s) is achieved than when cyclomethicone is used alone.

These results show that the absorption profile of at least one active can be controlled to the desired level by modulating the amount of the silicones in the formulation.

Throughout the specification, the term “means” in the context of means for carrying out a function, is intended to refer to at least one device adapted and/or constructed to carry out that function.

It will be appreciated that the invention is not restricted to the details described above with reference to the preferred embodiments but that numerous modifications and variations can be made without departing from the spirit or scope of the invention as defined by the following claims.

1. A therapeutic composition for topical application to skin comprising:
at least one active compound;
at least one penetration enhancer;
a penetration modulating component comprising at least one silicone; and
a fugitive solvent base.

2. The composition as claimed in claim 1 wherein the or at least one silicone is selected from the group consisting of dimethicones; cyclomethicones; simethicones; and oligodimethylsiloxanes.

3. The composition as claimed in claim 1 wherein the penetration modulating component is present in the composition in an amount of from about 10 wt % to about 70 wt % based on the total weight of the composition.

4. The composition as claimed in claim 1 wherein the penetration modulating component consists essentially of either a single silicone or a combination of two or three silicones.

5. The composition as claimed in claim 1 wherein Tₘₐₓ for said therapeutic composition is greater than Tₘₐₓ for a control composition consisting of all of the components of said therapeutic composition except for the penetration modulating component.

6. The composition as claimed in claim 1 wherein Cₛₛ for said therapeutic composition is at least substantially constant over at least 16 hours.

7. The composition as claimed in claim 1 wherein the penetration modulating component consists essentially of cyclomethicone and dimethicone.

8. The composition as claimed in claim 7 wherein cyclomethicone is present in an amount of from about 15 wt % to about 35 wt % based on the total weight of the composition.

9. The composition as claimed in claim 7 wherein dimethicone is present in an amount of from about 5 wt % to about 30 wt % based on the total weight of the composition.

10. The composition as claimed in claim 7 wherein cyclomethicone is present in an amount of about 30 wt % and dimethicone is present in an amount of about 10 wt %.

11. The composition as claimed in claim 1 wherein the penetration modulating component consists essentially of cyclomethicone and at least one oligo-dimethylsiloxane.

12. The composition as claimed in claim 11 wherein cyclomethicone is present in an amount of from about 15 wt % to about 25 wt % based on the total weight of the composition.

13. The composition as claimed in claim 11 wherein said oligodimethylsiloxane(s) is present in an amount of from about 5 wt % to about 20 wt % based on the total weight of the composition.

14. The composition as claimed in claim 11 wherein cyclomethicone is present in an amount of about 20 wt % and said oligodimethylsiloxane(s) is present in an amount of about 10 wt %.

15. The composition as claimed in claim 1 wherein the penetration modulating component consists essentially of dimethicone and at least one oligodimethylsiloxane.

16. The composition as claimed in claim 15 wherein dimethicone is present in an amount of from about 20 wt % to about 40 wt % based on the total weight of the composition.

17. The composition as claimed in claim 15 wherein said oligodimethylsiloxane(s) is present in an amount of from about 10 wt % to about 30 wt % based on the total weight of the composition.

18. The composition as claimed in claim 15 wherein dimethicone is present in an amount of about 30 wt % and said oligodimethylsiloxane(s) is present in an amount of about 20 wt %.

19. The composition as claimed in claim 1 wherein Cₘₐₓ for said therapeutic composition is greater than Cₘₐₓ for a control composition consisting of all of the components of said therapeutic composition except for the penetration modulating component.

20. The composition as claimed in claim 1 wherein the penetration modulating component consists essentially of cyclomethicone.

21. The composition as claimed in claim 19 wherein cyclomethicone is present in an amount from about 20 wt % to about 40 wt % based on the total weight of the composition.

22. The composition as claimed in claim 19 wherein cyclomethicone is present in an amount of about 30 wt %.

23. The composition as claimed in claim 1 wherein the penetration modulating component consists essentially of cyclomethicone, dimethicone and at least one oligodimethylsiloxane.

24. The composition as claimed in claim 23 wherein cyclomethicone is present in an amount from about 20 wt % to about 40 wt % based on the total weight of the composition.

25. The composition as claimed in claim 23 wherein dimethicone is present in an amount of from about 5 wt % to about 30 wt % based on the total weight of the composition.

26. The composition as claimed in claim 23 wherein said oligodimethylsiloxane(s) is present in an amount of from about 15 wt % to about 25 wt % based on the total weight of the composition.

27. The composition as claimed in claim 23 wherein cyclomethicone is present in an amount of about 30 wt %, dimethicone is present in an amount of about 10 wt % and said oligodimethylsiloxane(s) is present in about 20 wt %.

28. The composition as claimed in claim 1 wherein the or at least one active compound has a systemic effect.

29. The composition as claimed in claim 1 wherein the or at least one active compound is a pharmacologically active compound.

30. The composition as claimed in claim 29 wherein the pharmacologically active compound is nicotine.

31. The composition as claimed in claim 1 wherein the or at least one active compound is a nutraceutically active compound.

32. The composition as claimed in claim 1 wherein the composition is non-aqueous.

33. The composition as claimed in claim 1 wherein the or each penetration enhancer is a non-silicone penetration enhancer.

34. The composition as claimed in claim 1 wherein said penetration enhancer is benzyl alcohol.

35. The composition as claimed in claim 1 for use in the treatment of the human or animal body by therapy.

36. A dispenser comprising a container containing a therapeutic composition according to any of claim 1 and dispensing means for dispensing the composition.

37. A method of using a penetration modulating component comprising at least one silicone in a therapeutic composition for topical application to the skin comprising:
at least one active compound;
at least one penetration enhancer; and
a fugitive solvent base,
to increase at least one value selected from Tₘₐₓ, Cₘₐₓ, Cₛₛ and Tₛₛ for said therapeutic composition relative to a control composition consisting of each of the components of said therapeutic composition except for the penetration modulating component.

38. (canceled)

39. A method of preparing a therapeutic composition according to claim 1, said method comprising:
determining an appropriate release profile for the or each active component in view of the condition to be treated;
selecting an appropriate silicone or combination of silicones as said penetration modifying component; and
combining the or each active compound with at least one penetration enhancer, a fugitive solvent base and said penetration modifying component.

40. A therapeutic composition for topical application to skin comprising;
at least one active compound;
a penetration modulating component comprising at least one silicone; and
a fugitive solvent base.

41. A method of using at least one silicone in a therapeutic composition for topical application to skin comprising at least one active compound and a fugitive solvent base to modify transdermal penetration of the or each active compound relative to a corresponding composition without a penetration modulating component.

42.-45. (canceled)
